(19) United States
(12) Patent Application Publication
(10) Pub. No.: US 2024/0173162 A1
(43) Pub. Date: May 30, 2024

(54) FLUID COLLECTION DEVICE

(71) Applicant: PUREWICK CORPORATION, Covington, GA (US)

(72) Inventor: Camille Rose Newton, Bonsall, CA (US)

(21) Appl. No.: 18/549,387

(22) PCT Filed: Mar. 8, 2022
(86) PCT No.: PCT/US2022/019254

§ 371 Date: Sep. 7, 2023

Related U.S. Application Data
(60) Provisional application No. 63/159,186, filed on Mar. 10, 2021.

(51) Int. Cl. A61F 5/453; A61F 5/44

(52) U.S. Cl. CPC A61F 5/453; A61F 5/4404

(57) ABSTRACT

Examples relate to fluid collection devices having a fluid permeable body with enclosed edges, and related systems and methods. In some embodiments, a fluid collection device includes a tube having an inlet, a fluid permeable body, and a fluid impermeable barrier. The fluid permeable body has a first side, a second side, a proximal edge, and two side edges extending from the proximal edge towards the inlet. The fluid impermeable barrier covers the first side, the proximal edge, and the two side edges of the fluid permeable body effective to prevent fluid communication through the first side, the proximal edge, and the two side edges.

### CROSS-REFERENCE TO RELATED APPLICATIONS

This application claims priority to U.S. Provisional Patent Application No. 63/159,186 filed on Mar. 10, 2021, the disclosure of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters may be uncomfortable, painful, and may cause urinary tract infections.

Thus, users and manufacturers of fluid collection devices continue to seek new and improved devices, systems, and methods to collect urine.

### SUMMARY

Embodiments disclosed herein are related to fluid collection devices having a fluid permeable body with enclosed edges, and related systems and methods. In an embodiment, a fluid collection device includes a tube having an inlet, a fluid permeable body, a fluid impermeable barrier, and a chamber. The fluid permeable body has a first side, a second side, a proximal edge, and two side edges extending from the proximal edge towards the inlet. The fluid impermeable barrier covers the first side, the proximal edge, and the two side edges of the fluid permeable body effective to prevent fluid communication through the first side, the proximal edge, and the two side edges. The fluid impermeable barrier defines an aperture providing fluid communication between the fluid permeable body and at least a portion of the tube. The chamber is sized to receive at least a portion of a penis therein to position a portion of the penis adjacent to a portion of the fluid permeable body.

In an embodiment, a method of collecting urine includes positioning a penis on a first side of a fluid permeable body in a chamber of a urine collection device. The fluid permeable body has a second side opposite to the first side, a proximal edge, and two side edges extending from the proximal edge towards a distal end region of the urine collection device. The method also includes drawing urine through the fluid permeable body to an inlet of tube at the distal end region of the urine collection device. The second side, the proximal edge, and the two side edges of the fluid permeable body are covered by a fluid impermeable barrier such that the urine is drawn through the first side of the fluid permeable body positioned against the penis. The method also includes drawing at least some of the urine through the inlet of the tube and out of the urine collection device.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a block diagram of a fluid collection system, according to an embodiment.

FIG. 2A is a front view of a fluid collection device, according to an embodiment.

FIG. 2B is a cross-sectional view of the fluid collection device of FIG. 2A taken along line 2B-2B of FIG. 2A.

FIG. 3 is a cross-sectional view of a distal end region of a fluid collection device, according to an embodiment.

FIG. 4A is a front view of a fluid collection device, according to an embodiment.

FIG. 4B is a rear view of the fluid collection device of FIG. 4A.

FIG. 5 is a top view of a fluid collection device, according to an embodiment.

FIG. 6 is a bottom view of a fluid collection device, according to an embodiment.

FIG. 7A is a top view of a fluid collection device, according to an embodiment.

FIG. 7B is a bottom view of the fluid collection device of FIG. 7A.

FIG. 8 is a flow diagram of a method for assembling a portable urine collection system, according to an embodiment.

### DETAILED DESCRIPTION

Embodiments disclosed herein are related to fluid collection devices having a fluid permeable body with one or more edges, ends, and/or peripheries enclosed by a fluid impermeable barrier, and related systems and methods. In some embodiments, the fluid collection device is configured as a urine collection device, such as a male external catheter urine collection device. Fluid collection devices also may include wound or skin drainage devices that remove fluid from a wounds and/or the skin of a user. In one, some, or all embodiments of fluid collection devices described herein, having one or more edges, ends, and/or peripheries of a fluid permeable body enclosed by the fluid impermeable barrier results in the technical effect of fluid being pulled more efficiently into the fluid permeable body and directed to a sump and/or tube for removal from the fluid collection device. In at least one, some, or all embodiments of fluid collection devices described herein, the fluid permeable body has no edges, ends, and/or peripheries that are open to air and/or liquid flow because the fluid permeable barrier is enclosing each of the edges, ends, and/or peripheries. In at least one, some, or all embodiments of fluid collection devices described herein, having the edges, ends, and/or peripheries enclosed by the fluid impermeable barrier results in the technical effect of increasing the overall wicking capability of the fluid collection device, decreasing a loss of vacuum in the fluid collection device, decreasing fluid run off, and/or improving drawing of fluid through the fluid permeable body to the sump and tube for removal from the fluid collection device.

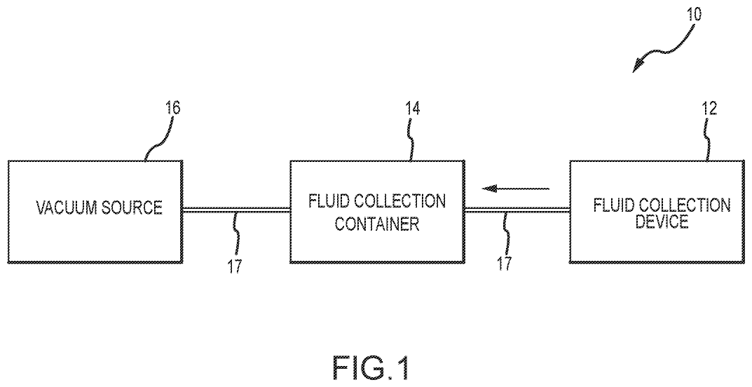

FIG. 1 is a block diagram of a fluid collection system 10, according to an embodiment. The fluid collection system 10 may be included in any of the embodiments of fluid collection systems described herein. The system 10 includes a fluid (e.g., urine) collection device 12 (e.g., any of the fluid collection assemblies disclosed herein), a fluid collection container 14, and a vacuum source 16 (e.g., pump). The fluid collection device 12, the fluid collection container 14, and the vacuum source 16 may be fluidly coupled to each other via one or more tubes 17 or conduits. For example, fluid collection device 12 may be operably coupled to one or more of the fluid collection container 14 or the vacuum source 16 via the tube 17. In some embodiments, the pump 16 may be secured directly to the fluid collection container 14. Fluid (e.g., urine or other bodily fluids) collected in the fluid collection device 12 may be removed from the fluid collection device 12 via the tube 17 secured to the fluid collection device 12. Suction force may be introduced into the chamber of the fluid collection device 12 via the inlet of the tube 17 responsive to suction (e.g., vacuum) force applied at the outlet of the tube 17.

The suction force may be applied to the outlet of the tube 17 by the vacuum source 16 either directly or indirectly. The suction force may be applied indirectly via the fluid collection container 14. For example, the outlet of the tube 17 may be disposed within or fluidly coupled to an interior region of the fluid collection container 14 and an additional tube 17 may extend from the fluid collection container 14 to the vacuum source 16. Accordingly, the vacuum source 16 may apply suction to the fluid collection device 12 via the fluid collection container 14. The suction force may be applied directly via the vacuum source 16. For example, the outlet of the tube 17 may be disposed within the vacuum source 16. An additional tube 17 may extend from the vacuum source 16 to a point outside of the fluid collection device 12, such as to the fluid collection container 14. In such examples, the vacuum source 16 may be disposed between the fluid collection device 12 and the fluid collection container 14.

The fluid collection container 14 is sized and shaped to retain a fluid therein. The fluid collection container 14 may include a bag (e.g., drainage bag), a bottle or cup (e.g., collection jar), or any other enclosed container for storing bodily fluid(s) such as urine. In some examples, the tube 17 may extend from the fluid collection device 12 and attach to the urine collection container 14 at a first point therein. An additional tube 17 may attach to the fluid collection container 14 at a second point thereon and may extend and attach to the vacuum source 16. Accordingly, a vacuum (e.g., suction) may be drawn through fluid collection device 12 via the fluid collection container 14. Fluid, such as urine, may be drained from the fluid collection device 12 using the vacuum source 16.

The vacuum source 16 may include one or more of a manual vacuum pump, and electric vacuum pump, a diaphragm pump, a centrifugal pump, a displacement pump, a magnetically driven pump, a peristaltic pump, or any source configured to produce a vacuum (e.g., negative pressure). The vacuum source 16 may provide a vacuum or suction to remove fluid from the fluid collection device 12. In some examples, the vacuum source 16 may be powered by one or more of a power cord (e.g., connected to a power socket), one or more batteries, or even manual power (e.g., a hand operated vacuum pump). In some examples, the vacuum source 16 may be sized and shaped to fit outside of, on, or within the fluid collection device 12. For example, the pump 16 may include one or more miniaturized pumps or one or more micro pumps. The vacuum sources disclosed herein may include one or more of a switch, a button, a plug, a remote, or any other device suitable to activate the vacuum source 16. Aspects of the fluid collection system 10 may be included in any of the fluid collection systems described herein. For example, the fluid collection device 12 may include any of the fluid or urine collection devices described herein.

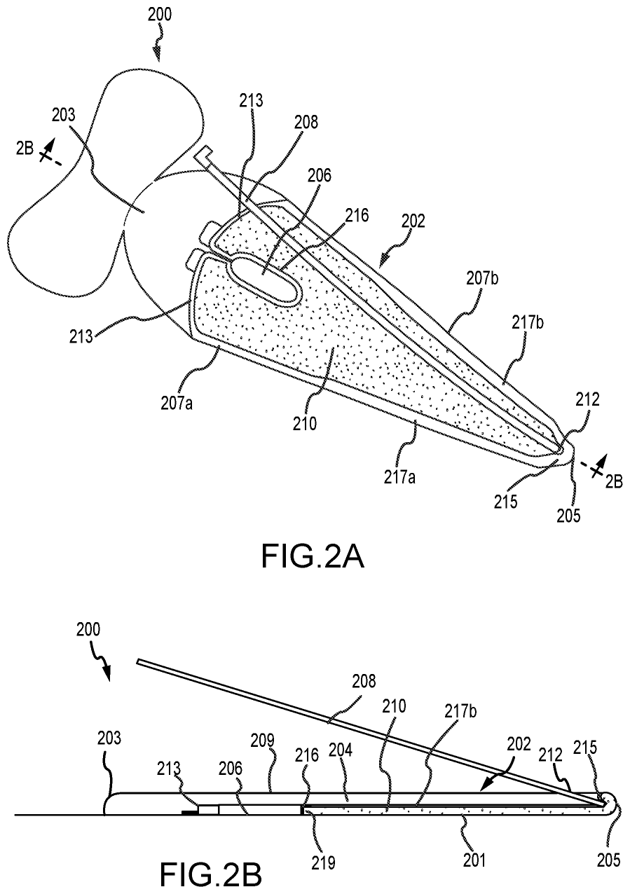

In many embodiments, the fluid collection device may be configured as a urine collection device. FIGS. 2-5, for example, are embodiments of male urine collection devices having a fluid permeable body with one or more edges, ends, and/or peripheries enclosed by a fluid impermeable barrier, and a chamber sized to receive at least a portion of a penis therein to position a portion of the penis adjacent to a portion of the fluid permeable body. As shall be described in greater detail below, embodiments of urine collection devices may include a tube having an opening, a fluid permeable body, a fluid impermeable barrier. The fluid permeable body according to different embodiments may include a first side, a second side, a proximal edge, and two side edges extending from the proximal edge towards the inlet of the tube. The fluid impermeable barrier may define an aperture providing fluid communication between the fluid permeable body and at least a portion of the tube. In some embodiments, the tube extends through the aperture in the fluid impermeable barrier. In some embodiments, the inlet of the tube is flush with the fluid impermeable barrier.

The fluid impermeable barrier according to different embodiments may cover the first side of the fluid permeable body, the proximal edge of the fluid permeable body, and the two side edges of the fluid permeable body effective to prevent fluid communication through the first side, the proximal edge, and the two side edges. The second side of the fluid permeable body may remain uncovered by the fluid impermeable barrier (e.g., the fluid impermeable barrier is absent from the second side of the fluid permeable body), resulting in the technical effect of drawing urine discharged by the penis into the fluid permeable body through the second side of the fluid permeable body in at least one, some, or all embodiments of the fluid collection devices described herein. In covering the portions of the fluid permeable described herein, the fluid impermeable barrier may be adjacent to and/or interfacing the reference portion of the fluid permeable body. For example, the fluid impermeable barrier may be adjacent to and/or interfacing the first side of the fluid permeable body, the proximal edge of the fluid permeable body, and the two side edges of the fluid permeable body. In some embodiments, the fluid impermeable barrier may be secured to the referenced portion(s) of the fluid permeable body such that the fluid impermeable barrier is covering through one or more of adhesives, a press fit, molding, or combinations thereof. For example, the fluid impermeable barrier may be secured to the first side of the fluid permeable body, the proximal edge of the fluid permeable body, and the two side edges of the fluid permeable body.

FIG. 2A is a front or top view of a male urine collection device 200, and FIG. 2B is a cross-sectional view of the urine collection device 200 taken along line 2B-2B of FIG. 2A, according to an embodiment. The urine collection device 200 includes a fluid impermeable barrier 202 at least partially defining an opening 206 and a chamber 204 within the fluid collection device and in fluid communication with the opening 206. In some embodiments, a bottom portion 201 of the fluid impermeable barrier 202 may define the opening 206 and an opposite or a top portion 209 of the fluid impermeable barrier may defined an aperture 212. In the urine collection device 200, the opening 206 is defined by the bottom portion 201 of the fluid impermeable barrier 202 and visible through the transparent top portion 209 of the fluid impermeable barrier 202. The fluid impermeable barrier 202 includes a proximal end region 203 and a distal end region 205. The opening 206 may be positioned proximate or closer to the proximal end region 203 than the distal end region 205 of the fluid impermeable barrier 202. The chamber 204 may be defined by the top portion 209 and the bottom portion 201 of the fluid impermeable barrier 202 may extend between the proximal end region 203 of the fluid impermeable barrier 202 and the distal end region 205 of the fluid impermeable barrier 202. In some embodiments, the fluid impermeable barrier 202 includes edges 207a, 207b that narrow closer together between the proximal end region 203 and the distal end region 205. For example, the fluid impermeable barrier 202 (and the chamber 204) may include a substantially triangular front profile, with the distal end region 205 being at the narrow end or tip of the triangular profile. The fluid impermeable barrier 202 may include a substantially flexible fluid impermeable material, such as a fluid impermeable polymer (e.g., silicone, polypropylene, polyethylene, polyethylene terephthalate, a polycarbonate, etc.), polyurethane films, thermoplastic elastomer, oil, another suitable material, or combinations thereof. In some embodiments, the fluid impermeable barrier includes a paper-like fluid impermeable material or a fluid impermeable fabric.

The urine collection device 200 also includes a fluid permeable body 210 positioned within the chamber 204 and extending at least partially between the distal end region 205 and the proximal end region 203. The fluid permeable body 210 may be shaped generally complementary to the shape of the chamber 204 of the fluid impermeable barrier 202. The fluid permeable body 210 may include a distal portion positioned in or proximate to the distal end region 205 of the fluid impermeable body 202. The fluid permeable body 210 also includes a proximal edge distal to the distal portion of the fluid permeable body 210 and two side edges extending between the proximal edge and the distal portion of the chamber 204 210. As shall be described in greater detail below, the proximal edge of the fluid permeable body 210 may be positioned in the proximal end region 203 of the fluid impermeable barrier 202, such as within a slot defined by a proximal edge portion 213 of the fluid impermeable barrier 202. The side edges of the fluid permeable body 210 may be positioned proximate to the side edges 207a, 207b of the fluid impermeable barrier 202, such as within a side edge slot defined by a slide edge portion 217a, 217b of the fluid impermeable barrier 202. In some embodiments, the distal portion of the fluid permeable body 210 may be positioned within a slot defined by a distal edge portion 215 of the fluid impermeable barrier 202.

The fluid permeable body 210 can be configured to wick and/or allow transport of fluid away from the opening 206, thereby preventing the fluid from escaping the chamber 204. The fluid permeable body 210 also can wick and/or allow transport of the fluid generally towards the sump. The fluid permeable body 210 can include any material that can wick and/or allow transport of the fluid. The permeable properties referred to herein can be wicking, capillary action, diffusion, or other similar properties or processes, and are referred to herein as “permeable” and/or “wicking.” Such “wicking” or other physical properties may exclude absorption into the fluid permeable body 210, such as not include adsorption of the bodily fluids into the fluid permeable body 210. Put another way, substantially no absorption or solubility of the bodily fluids into the material may take place after the material is exposed to the bodily fluids and removed from the bodily fluids for a time. While no absorption or solubility is desired, the term “substantially no absorption” may allow for nominal amounts of absorption and/or solubility of the bodily fluids into the fluid permeable body 210 (e.g., absorbency), such as less than about 30 wt % of the dry weight of the fluid permeable body 210, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the fluid permeable body 210. In an embodiment, the fluid permeable body 210 may include at least one absorbent or adsorbent material.

The fluid permeable body 210 can include a one-way fluid movement fabric. As such, the fluid permeable body 210 can remove fluid from the area around the penis, thereby leaving the area and urethra dry. The fluid permeable body 210 can enable the fluid to flow generally towards the sump and the tube 208 within the chamber 204. The fluid permeable body 210 can include a porous or fibrous material, such as hydrophilic polyolefin. In some embodiments, the fluid permeable body 210 consists of or consists essentially of a porous or fibrous material, such as hydrophilic polyolefin. Examples of polyolefin that can be used in the fluid permeable body 210 include, but are not limited to, polyethylene, polypropylene, polyisobutylene, ethylene propylene rubber, ethylene propylene diene monomer, or combinations thereof. Moreover, the fluid permeable body 210 can be manufactured according to various manufacturing methods, such as molding, extrusion, or sintering. The fluid permeable body 210 can include varying densities or dimensions.

In some embodiments, the fluid permeable body 210 can include two or more layers of fluid permeable materials. For example, the fluid permeable body 210 can include a fluid permeable membrane covering or wrapped around a fluid permeable support, with both the fluid permeable membrane and the fluid permeable support being disposed in the chamber 204. The fluid permeable membrane can cover or extend across at least a portion (e.g., all) of at least the side of the fluid permeable support facing the penis of the user. The fluid permeable membrane and the fluid permeable support can be configured to wick any fluid away from the opening 206, thereby preventing the fluid from escaping the chamber 204 and promoting removal of the fluid through the tube 208. The permeable properties referred to herein can be wicking, capillary action, diffusion, or other similar properties or processes, and are referred to herein as “permeable” and/or “wicking.”

The fluid permeable membrane and the fluid permeable support also can wick and/or allow transport of the fluid generally towards an interior of the chamber 204, such as the sump. The fluid permeable membrane can include any material that can wick the fluid. For example, the fluid permeable membrane can include fabric, such as a gauze (e.g., a silk, linen, polymer based materials such as polyester, or cotton gauze), nylon (such as a spun nylon fibers), another soft fabric (e.g., jersey knit fabric or the like), or another smooth fabric (e.g., rayon, satin, or the like). Forming the fluid permeable membrane from gauze, soft fabric, and/or smooth fabric can reduce chaffing caused by the urine collection device 200. Other embodiments of fluid permeable membranes and fluid permeable supports are disclosed in U.S. patent application Ser. No. 15/612,325 filed on Jun. 2, 2017; U.S. patent application Ser. No. 15/260,103 filed on Sep. 8, 2016; U.S. patent application Ser. No. 15/611,587 filed on Jun. 1, 2017; PCT Patent Application No. PCT/US19/29608, filed on Apr. 29, 2019, the disclosure of each of which is incorporated herein, in its entirety, by this reference. In many embodiments, the fluid permeable body 210 includes a fluid permeable support including a porous spun nylon fiber structure and a fluid permeable wicking membrane including gauze at least partially enclosing the spun nylon fiber structure. For example, the fluid permeable body 210 may include a gauze or other wicking fabric positioned to contact the skin of the user. In some embodiments, the gauze or other wicking fabric is wrapped around a body of spun nylon fibers material and/or covering both sides of a substantially planar spun nylon fibers material. In some embodiments, the gauze or other wicking fabric covers the side of substantially planar spun nylon fibers material that is oriented towards the skin of the user.

The urine collection device 200 also includes a tube 208 extending into the chamber 204 and having an end positioned proximate to the distal end region 205 of the fluid impermeable barrier 202. The tube 208 includes a tube opening or inlet proximate to the end of the tube 208. At least some of the fluid permeable body 210 may extend into a sump region or area between the inlet and the distal end region 205 of the fluid impermeable barrier 202. Fluid discharged on the fluid permeable body 210 may flow to and pool in the sump region for removal when a vacuum is applied on the tube 208. The tube 208 may extend through an aperture 212 in a top portion 209 of the fluid impermeable barrier 202 and into the chamber 204. The aperture 212 may be positioned proximate to the distal end region 205 of fluid impermeable barrier 202. In some embodiments, the end of the tube 208 and/or the inlet are positioned between the distal end region 205 and the aperture 212. In some embodiments, the aperture 212 is absent, and the tube 208 may exit the chamber 204 through the opening 206. U.S. Provisional Patent 63/067,542 describes embodiments of male external catheter urine collection devices, aspects of which may be used in any of the embodiments disclosed herein, the disclosure of which are incorporated in their entirety by this reference.

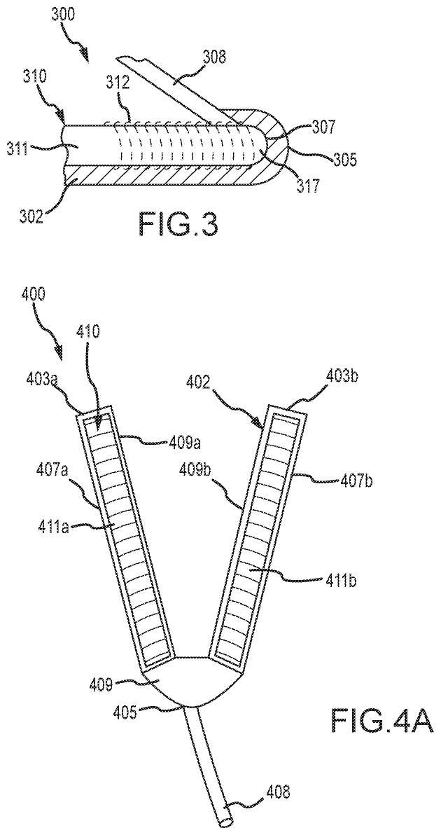

One or more edges, ends, and/or peripheries of the fluid permeable body 210 are enclosed by portions of the fluid impermeable barrier 202. Enclosing the edges, ends, and/or peripheries of the fluid permeable body 210 with portions of the fluid impermeable barrier 202 creates a barrier around the edges to prevent air, vacuum, and liquid loss, while still maximizing fluid permeable body 210 contact with skin and fluid. In some embodiments, the fluid impermeable barrier 202 may include a proximal edge portion 213 defining a proximal slot enclosing a proximal edge of the fluid permeable body 210. Although the proximal slot defined by the proximal edge portion 213 and the proximal edge of the fluid permeable body 210 are not visible in FIGS. 2A and 2B, the proximal slot defined by the proximal edge portion 213 and the proximal edge of the fluid permeable body 210 may be similar to or the same as the slot 307 defined by the distal end region 305 of the fluid impermeable barrier 302 and the distal edge 317 of the fluid permeable body 310 shown in FIG. 3, and described below. The proximal edge portion 213 may enclose at least some of the top side or surface of the fluid permeable body 210 within the proximal slot. In some embodiments, the proximal edge portion 213 extends continuously from the first edge 207a to the second edge 207b of the fluid impermeable barrier 202. In some embodiments, the proximal edge portion 213 encloses or covers the entirety of proximal edge of the fluid permeable body 210 with the proximal slot. In some embodiments, two portions of the fluid permeable body 210 may terminate between the opening 206 and the distal end region 203 of the chamber 204, and the proximal edge portion 213 may enclose or cover all of the terminating edge of the fluid permeable body 210 within the proximal slot.

The proximal edge portion 213 of the fluid impermeable barrier 202 may be secured to and/or extend from at least one of the bottom portion 201 and/or the top portion 209 of the fluid impermeable barrier 202. In some embodiments, the proximal edge portion 213 is separate from the top portion 209 and the bottom portion 201 of the fluid impermeable barrier 202, and is secured to the proximal edge of the fluid permeable body 210. The proximal edge portion 213 may include any of the fluid impermeable materials described above in relation to the fluid impermeable barrier. In some embodiments, the proximal edge portion includes a polyvinyl chloride foam. The polyvinyl chloride foam may be secured to the fluid permeable body 210 with an acrylate adhesive.

When a penis is inserted into the chamber 204 of the fluid impermeable barrier 202 through the opening 206, the penis may space at least some of the top portion 209 of the fluid impermeable barrier 202 from the front or top side of the fluid permeable body 210, visible in FIG. 2A. When the penis is positioned within the chamber 204, the back side (shown in FIG. 2B) of the fluid permeable body 210 is covered by the bottom (or back) portion 201 of the fluid impermeable barrier 202. In some embodiments, the back side of the fluid permeable body 210 is secured to the bottom portion 201 of the fluid impermeable barrier 202. Although a gap or space is formed between at least some of the top portion 209 of the fluid impermeable barrier 202 and the front or top side of the fluid permeable body 210 when a penis is positioned within the chamber 204, the proximal edge portion 213 is configured to remain covering the proximal edge of the fluid permeable body 210 and at least some of the front side of the fluid permeable body 210.

In some embodiments, the fluid impermeable barrier 202 may include an opening or additional edge portion 216 defining a slot 219 (shown in FIG. 2B) enclosing an edge of the fluid permeable body 210 that at least partially surrounds the opening 206. The slot 219 defined by the opening edge portion 216 and the edge of the fluid permeable body 210 at least partially surrounding the opening 206 may be similar to or the same as the slot 307 defined by the distal end region 305 of the fluid impermeable barrier 302 and the distal edge 317 of the fluid permeable body 310 shown in FIG. 3, and described below. The opening edge portion 216 may enclose at least some of the top side or surface of the fluid permeable body 210 within the slot 219. In some embodiments, the opening edge portion 216 extends continuously around and surrounds the opening 206. In some embodiments, the opening edge portion 216 extends only part way around the opening 206.

The opening edge portion 216 of the fluid impermeable barrier 202 may be secured to and/or extend from the bottom portion 201 of the fluid impermeable barrier 202. In some embodiments, the opening edge portion 216 is separate from the top portion 209 and the bottom portion 201 of the fluid impermeable barrier 202, and is secured to the edge of the fluid permeable body 210 at least partially surrounding the opening 206. The opening edge portion 216 may include any of the fluid impermeable materials described above in relation to the fluid impermeable barrier. In some embodiments, the opening edge portion 216 includes a polyvinyl chloride foam. The polyvinyl chloride foam may be secured to the fluid permeable body 210 with an acrylate adhesive.

When a penis is inserted into the chamber 204 of the fluid impermeable barrier 202 through the opening 206, the penis may space at least some of the top portion 209 of the fluid impermeable barrier 202 from the front or top side of the fluid permeable body 210, visible in FIG. 2A. Although a gap or space is formed between at least some of the top portion 209 of the fluid impermeable barrier 202 and the front or top side of the fluid permeable body 210 when a penis is positioned within the chamber 204, the opening edge portion 216 is configured to remain covering the edge of the fluid permeable body 210 surrounding the opening 206 and at least some of the front side of the fluid permeable body 210.

In some embodiments, the fluid impermeable barrier 202 may include side edge portions 217a, 217b defining side slots enclosing side edges of the fluid permeable body 210. Although the side slots defined by the side edge portions 217a, 217b and the side edges of the fluid permeable body 210 are not visible in FIGS. 2A and 2B, the side slots defined by the side edge portions 217a, 217b and the side edge of the fluid permeable body 210 may be similar to or the same as the slot 307 defined by the distal end region 305 of the fluid impermeable barrier 302 and the distal edge 317 of the fluid permeable body 310 shown in FIG. 3, and described below. The side edge portions 217a, 217b may enclose at least some of the top side or surface of the fluid permeable body 210 within the side slots. In some embodiments, the side edge portions 217a, 217b extend continuously from the proximal edge portion 213 of the fluid impermeable barrier 202 to the distal end region 205 of the fluid impermeable barrier 202.

The side edge portions 217a, 217b of the fluid impermeable barrier 202 may be secured to and/or extend from at least one of the bottom portion 201 and/or the top portion 209 of the fluid impermeable barrier 202. In some embodiments, the side edge portions 217a, 217b are separate from the top portion 209 and the bottom portion 201 of the fluid impermeable barrier 202, and are secured to the side edges of the fluid permeable body 210. The side edge portions 217a, 217b may include any of the fluid impermeable materials described above in relation to the fluid impermeable barrier 202. In some embodiments, the side edge portions 217a, 217b include a polyvinyl chloride foam. The polyvinyl chloride foam may be secured to the fluid permeable body 210 with an acrylate adhesive.

When a penis is inserted into the chamber 204 of the fluid impermeable barrier 202 through the opening 206, the penis may space at least some of the top portion 209 of the fluid impermeable barrier 202 from the front or top side of the fluid permeable body 210, visible in FIG. 2A. Although a gap or space is formed between at least some of the top portion 209 of the fluid impermeable barrier 202 and the front or top side of the fluid permeable body 210 when a penis is positioned within the chamber 204, the side edge portions 217a, 217b are configured to remain covering the side edges of the fluid permeable body 210 and at least some of the front side of the fluid permeable body 210.

In some embodiments, the fluid impermeable barrier 202 may include a distal edge portion 215 defining a distal slot enclosing a distal edge of the fluid permeable body 210. Although the distal slot defined by the distal edge portion 215 and the distal edge of the fluid permeable body 210 are not visible in FIG. 2A, the distal slot defined by the distal edge portion 215 and the distal edge of the fluid permeable body 210 may be similar to or the same as the slot 307 defined by the distal end region 305 of the fluid impermeable barrier 302 and the distal edge 317 of the fluid permeable body 310 shown in FIG. 3, and described below. The distal edge portion 215 may enclose at least some of the top side or surface of the fluid permeable body 210 within the distal slot.

The distal edge portion 215 of the fluid impermeable barrier 202 may be secured to and/or extend from at least one of the bottom portion 201 and/or the top portion 209 of the fluid impermeable barrier 202. In some embodiments, the distal edge portion 215 is separate from the top portion 209 and the bottom portion 201 of the fluid impermeable barrier 202, and is secured to the distal edge of the fluid permeable body 210. The distal edge portion 215 may include any of the fluid impermeable materials described above in relation to the fluid impermeable barrier 202. In some embodiments, the distal edge portion 215 includes a polyvinyl chloride foam. The polyvinyl chloride foam may be secured to the fluid permeable body 210 with an acrylate adhesive.

When a penis is inserted into the chamber 204 of the fluid impermeable barrier 202 through the opening 206, the penis may space at least some of the top portion 209 of the fluid impermeable barrier 202 from the front or top side of the fluid permeable body 210, visible in FIG. 2A. Although a gap or space is formed between at least some of the top portion 209 of the fluid impermeable barrier 202 and the front or top side of the fluid permeable body 210 when a penis is positioned within the chamber 204, the distal edge portion 215 is configured to remain covering the distal edge of the fluid permeable body 210 and at least some of the front side of the fluid permeable body 210.

Turning to FIG. 3, a cross-sectional view of a distal end region of a urine collection device 300 is shown. The cross-sectional view shown in FIG. 3 may be similar to or the same as a cross-sectional view of the urine collection device 200, with the top portion 209 and the bottom portion 201 of the fluid impermeable barrier 202 removed. In some embodiments, the urine collection device may include a fluid impermeable barrier 302 and a fluid permeable body 310. Unless otherwise noted, the fluid impermeable barrier 302 may include any aspect of the fluid impermeable barrier 202 and/or the edge portions 213, 215, 216, 217a, 217b of the fluid impermeable barrier 202 described above. In some embodiments, the fluid impermeable barrier 302 includes a distal edge portion 315 defining a distal slot 307 sized to receive the fluid permeable body 310 therein.

The fluid permeable body 310 may include any aspect of the fluid permeable body 210 described above. For example, the fluid permeable body 310 may include a fluid permeable porous fibers structure 311 and an outer fluid permeable wicking material 312. The outer fluid permeable wicking material 312 may be positioned to contact the penis when the urine collection device 300 is in use. In some embodiments, the outer fluid permeable wicking material 312 surrounds or wraps around the fluid permeable porous fibers structure 311. In some embodiments, the outer fluid permeable wicking material 312 covers only the top side of the fluid permeable porous fibers structure 311.

The fluid permeable body 310 includes a distal edge 317 positioned within the distal slot 307 defined by the distal edge portion 305 of the fluid impermeable barrier 302. In some embodiments, the distal edge 317 is enclosed within the distal slot 307 such that the distal edge portion 305 of the fluid impermeable barrier 305 extends around the distal edge 317 to cover at least a portion of the top side of the fluid permeable body 310. In some embodiments, the distal edge portion 305 of the fluid impermeable barrier 302 may extend onto the top side of the fluid permeable body 310 until the distal edge portion 305 intersects the tube 308. The distal edge portion 305 may be adjacent to and/or interfacing the top side of the fluid permeable body 310, the distal edge 317 of the fluid permeable body 310, and the bottom side of the fluid permeable body 310. In some embodiments, the distal edge portion 305 may be secured to the distal edge 305 of the fluid permeable body 310 through one or more of adhesives, a press fit, molding, or combinations thereof.

In some embodiments, the end of the tube 308 includes a beveled end with the inlet positioned at the beveled end. The beveled end of the tube 308 may be at an acute or obtuse angle relative to the sidewall of the tube 308. With the beveled end being angled relative to the sidewall of the tube 308, when the beveled end and the inlet are generally horizontal, the tube 308 may angle relative to the fluid permeable body 310 and the urine collection device 300 may be used in a substantially horizontal position.

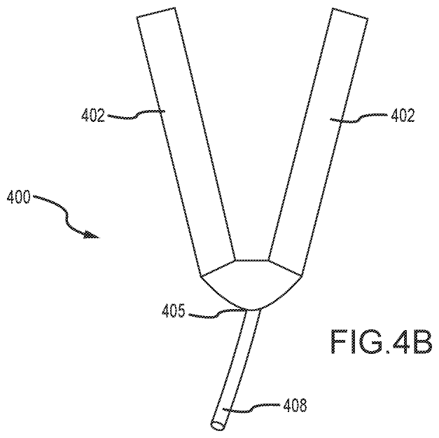

FIG. 4A is a front view of urine collection device 400 and FIG. 4B is a rear view of the urine collection device 400, according to an embodiment. Although referenced as a urine collection device 400, the urine collection device 400 also may be used in other fluid collection applications on the body of a user, such as wound drainage or fluid collection from the skin of the user. In some embodiments, the urine collection device 400 may be used without or in the absence of other materials or elements. In some embodiments, the urine collection device 400 may be positioned within the chamber defined by the fluid impermeable barrier 202 described above in relation to FIGS. 2A and 2B, and replace the fluid permeable body 210 shown in FIG. 4A. The urine collection device 400 may include a fluid impermeable barrier 402 and a fluid permeable body 410. Unless otherwise noted, the fluid permeable body 410 may include any aspect of the fluid permeable bodies 210, 310 described above and the fluid impermeable barrier 402 may include any aspect of the fluid impermeable barrier 202, 302 described above.

In some embodiments, the fluid permeable body 410 includes a distal (or intermediate) portion and two or more arms 411a, 411b extending from the distal portion of the fluid permeable body 410. In the urine collection device 400, the distal portion of the fluid permeable body 410 is covered by a distal edge portion 409 of the fluid impermeable barrier 402. The distal portion of the fluid permeable body 410 is in fluid communication with each of the arms 411a, 411b of the fluid permeable body 410. Each of the two or more arms 411a, 411b may include a terminating edge, an outer edge extending between the terminating edge and the distal portion of the fluid permeable body 410, an inner edge extending between the terminating edge and the distal portion of the fluid permeable body 410, a front side or surface, and a back side or surface. In FIG. 4A, the front side or surface of each arm 411a, 411b is exposed and visible. In FIG. 4B, the back side or surface of the fluid permeable body 410 is covered by the fluid impermeable barrier 402.

One or more edges, ends, and/or peripheries of the fluid permeable body 410 are enclosed by portions of the fluid impermeable barrier 402. Enclosing the edges, ends, and/or peripheries of the fluid permeable body 410 with portions of the fluid impermeable barrier 402 creates a barrier around the edges to prevent air, vacuum, and liquid loss, while still maximizing fluid permeable body 410 contact with skin and fluid. In some embodiments, the fluid impermeable barrier 402 may include a proximal edge portions 403a, 403b defining a proximal slot enclosing a terminating edge of each arm 411a, 411b of the fluid permeable body 410. Although the proximal slots defined by the proximal edge portions 403a, 403b and the terminating edges of each arm 411a, 411b of the fluid permeable body 410 are not visible in FIG. 4A, the proximal slot defined by the proximal edge portions 403a, 403b and the terminating edges of the arms 411a, 411b of the fluid permeable body 410 may be similar to or the same as the slot 307 defined by the distal end region 305 of the fluid impermeable barrier 302 and the distal edge 317 of the fluid permeable body 310 shown in FIG. 3, and described above. The proximal edge portions 403a, 403b may enclose at least some of the top side or surface of the fluid permeable body 410 within the proximal slot. In some embodiments, the proximal edge portions 403a, 403b encloses or covers the entirety of terminating edges of the arms 411a, 411b of the fluid permeable body 410 with the proximal slot. The proximal edge portions 403a, 403b may include any of the fluid impermeable materials described above in relation to the fluid impermeable barrier. In some embodiments, the proximal edge portions 403a, 403b include a polyvinyl chloride foam. The polyvinyl chloride foam may be secured to the fluid permeable body 410 with an acrylate adhesive.

In some embodiments, the fluid impermeable barrier 402 may include side edge portions 407a, 407b defining side slots enclosing the outer side edges of the arms 411a, 411b of the fluid permeable body 410. Although the side slots defined by the side edge portions 407a, 407b and the outer side edges of the arms 411a, 411b of the fluid permeable body 410 are not visible in FIG. 4A, the side slots defined by the side edge portions 407a, 407b and the outer side edges of the arms 411a, 411b of the fluid permeable body 410 may be similar to or the same as the slot 307 defined by the distal end region 305 of the fluid impermeable barrier 302 and the distal edge 317 of the fluid permeable body 310 shown in FIG. 3, and described above. The side edge portions 407a, 407b may enclose at least some of the top side or surface of the arms 411a, 411b within the side slots. In some embodiments, the side edge portions 407a, 407b extend continuously from the proximal edge portion 403a, 403b of the fluid impermeable barrier 402 to the distal end region 405 of the fluid impermeable barrier 402. The side edge portions 407a, 407b may include any of the fluid impermeable materials described above in relation to the fluid impermeable barrier 402. In some embodiments, the side edge portions 407a, 407b include a polyvinyl chloride foam. The polyvinyl chloride foam may be secured to the fluid permeable body 410 with an acrylate adhesive.

In some embodiments, the fluid impermeable barrier 402 may include side edge portions 409a, 409b defining side slots enclosing the inner side edges of the two or more arms 411a, 411b of the fluid permeable body 410. Although the side slots defined by the side edge portions 409a, 409b and the inner side edges of the arms 411a, 411b of the fluid permeable body 410 are not visible in FIG. 4A, the side slots defined by the side edge portions 409a, 409b and the inner side edges of the arms 411a, 411b of the fluid permeable body 410 may be similar to or the same as the slot 307 defined by the distal end region 305 of the fluid impermeable barrier 302 and the distal edge 317 of the fluid permeable body 310 shown in FIG. 3, and described above. The side edge portions 409a, 409b may enclose at least some of the top side or surface of the arms 411a, 411b within the side slots. In some embodiments, the side edge portions 409a, 409b extend continuously from the proximal edge portion 403a, 403b of the fluid impermeable barrier 402 to the distal end region 405 of the fluid impermeable barrier 402. The side edge portions 409a, 409b may include any of the fluid impermeable materials described above in relation to the fluid impermeable barrier 402. In some embodiments, the side edge portions 409a, 409b include a polyvinyl chloride foam. The polyvinyl chloride foam may be secured to the fluid permeable body 410 with an acrylate adhesive. In some embodiments, the side edge portion 409a, 409b may be absent to allow urine to be drawn into the inner side edges of the fluid permeable body 410.

While the arms 411a, 411b are angled relative to one another, in some embodiments, the urine collection device 400 may include multiple arms 411a, 411b that are substantially parallel to on another. Arms 411a, 411b that are substantially parallel to another may be spaced from one another or may abut/interface adjacent arm(s) of the arms 411a, 411b. In some embodiments, the urine collection device 400 may include three, four, five, or more arms 411 substantially parallel to one another. The side edge portions 407a, 407b, 409a, 409b may form channels for fluid collected in the arms 411a, 411b to flow through. In some embodiments, each of the arms 411a, 411b may include a spun nylon fibers material, and a single sheet of wicking material (such as gauze) may overlay all of the arms 411a, 411b. In some embodiments, wicking material completely surrounds the spun plastic layer in each arm 411a, 411b.

In some embodiments, the fluid impermeable barrier 402 may include a distal edge portion 409 and a distal end region 405 of the fluid impermeable barrier 402 defining a distal slot enclosing the distal portion of the fluid permeable body 410. Although the distal slot defined by the distal edge portion 409 and the distal portion of the fluid permeable body 410 are not visible in FIGS. 4A and 4B, the distal slot defined by the distal edge portion 409 and the distal edge of the fluid permeable body 410 may be similar to or the same as the slot 307 defined by the distal end region 305 of the fluid impermeable barrier 302 and the distal edge 317 of the fluid permeable body 310 shown in FIG. 3, and described above. The distal edge portion 409 may enclose and cover at least some (e.g., all) of the top side and the bottom side of the fluid permeable body 410 within the distal slot.

The urine collection device 400 also may include a tube 408 in fluid communication with the distal portion of the fluid permeable body 410. In some embodiments, the tube 408 extends through the distal end region 405 of the fluid impermeable barrier and the tube opening or inlet of the tube 408 is positioned within the distal portion of the fluid permeable body 410. In some embodiments, the inlet is generally flush with the distal end region 405 of the fluid impermeable barrier 402 and in fluid communication with the distal portion of the fluid permeable body 410.

FIG. 4B shows the back side of the urine collection device 400. In some embodiments, the fluid impermeable barrier 402 covers at least a portion (e.g. all) of the back side of the arms 411a, 411b and the distal portion of the fluid permeable body 410.

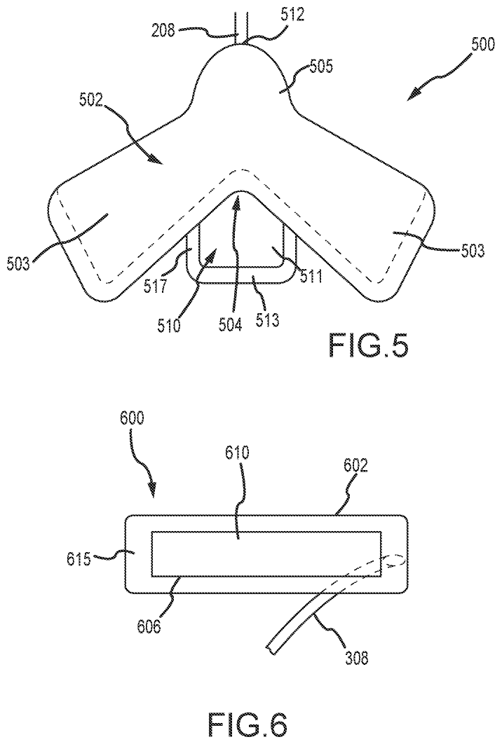

FIG. 5 is a top view of a urine collection device 500, according to an embodiment. The urine collection device may include a fluid impermeable barrier 502 and a fluid permeable body 510. Unless otherwise noted, the fluid impermeable barrier 502 may include any aspect of the fluid impermeable barriers 202, 302, 402 and the fluid permeable body 410 may include any aspect of the fluid permeable bodies 210, 310, 410.

In some embodiments, the fluid permeable body 510 includes a cup-shaped distal portion having an outer (or first) side and an inner (or second) side. In FIG. 5, the outer side of the cup-shaped distal portion of the fluid permeable body is covered by the cup-shaped distal end region 505 of the fluid impermeable barrier 502. The inner side of the cup-shaped distal portion of the fluid permeable body 510 may at least partially define a chamber 504 sized to receive at least a portion (e.g., the head) of the penis therein to positioned the portion of the head at least proximate (e.g., adjacent) to the inner side of the cup-shaped distal portion of the fluid permeable body 510.

The fluid permeable body also may include multiple tabs 511 extending from and in fluid communication with the distal portion of the fluid permeable body 510. While the urine collection device 500 includes three tabs 511 of the fluid permeable body 510, other embodiments may include four, five, six, etc., tabs. The multiple tabs 511 may be positioned circumferentially around the distal portion of the fluid permeable body 510. In some embodiments, edges of the tabs 511 may intersect at distal portion of the fluid permeable body 510. Each of the tabs includes an outer (or first) side and an inner (or second) side. In FIG. 5, a portion of the inner side of the center tab 511 is visible, while the outer sides of the right and left tabs 511 are covered by the fluid impermeable barrier 502.

The fluid impermeable barrier 502 may include a distal end region 505 covering the outer side of the cup-shaped distal portion of the fluid permeable body 510. The distal end region 505 may define an aperture 512 and the tube 208 may extend through or have fluid communication with the chamber 504 through the aperture 512. The fluid impermeable barrier 502 also may include multiple tabs 503 covering the outer side of the tabs 511 of the fluid permeable body 510. The fluid impermeable barrier 502 also may enclose or cover one or more edges, ends, and/or peripheries of the tabs 511 of the fluid permeable body 510. In some embodiments, the fluid impermeable barrier 502 may include proximal edge portions 513 defining a proximal slot enclosing a terminating edge of each tab 511 of the fluid permeable body 510. Although the proximal slots defined by the proximal edge portions 513 and the terminating edges of each tab 511 of the fluid permeable body 510 are not visible in FIG. 5, the proximal slot defined by the proximal edge portions 513 and the terminating edges of the tabs 511 of the fluid permeable body 510 may be similar to or the same as the slot 307 defined by the distal end region 305 of the fluid impermeable barrier 302 and the distal edge 317 of the fluid permeable body 310 shown in FIG. 3, and described above. The proximal edge portions 513 may enclose at least some of the inner side or surface of the fluid permeable body 510 within the proximal slot. In some embodiments, the proximal edge portions 513 enclose or cover the entirety of terminating edges of the tabs 511 of the fluid permeable body 510 with the proximal slots. The proximal edge portions 513 may include any of the fluid impermeable materials described above in relation to the fluid impermeable barrier. In some embodiments, the proximal edge portions 513 include a polyvinyl chloride foam. The polyvinyl chloride foam may be secured to the fluid permeable body 510 with an acrylate adhesive.

In some embodiments, the fluid impermeable barrier 502 may include side edge portions 517 defining side slots enclosing the side edges of the tabs 511 of the fluid permeable body 510. Although the side slots defined by the side edge portions 517 and the outer side edges of the tabs 511 of the fluid permeable body 510 are not visible in FIG. 5, the side slots defined by the side edge portions 517 and the outer side edges of the tabs 511 of the fluid permeable body 510 may be similar to or the same as the slot 307 defined by the distal end region 305 of the fluid impermeable barrier 302 and the distal edge 317 of the fluid permeable body 310 shown in FIG. 3, and described above. The side edge portions 517 may enclose at least some of the inner side or surface of the tabs 511 within the side slots. In some embodiments, the side edge portions 511 extend continuously from the proximal edge portion 513 of the fluid impermeable barrier 502 to the distal end region 505 of the fluid impermeable barrier 502. The side edge portions 517 may include any of the fluid impermeable materials described above in relation to the fluid impermeable barrier 502. In some embodiments, the side edge portions 517 include a polyvinyl chloride foam. The polyvinyl chloride foam may be secured to the fluid permeable body 510 with an acrylate adhesive.

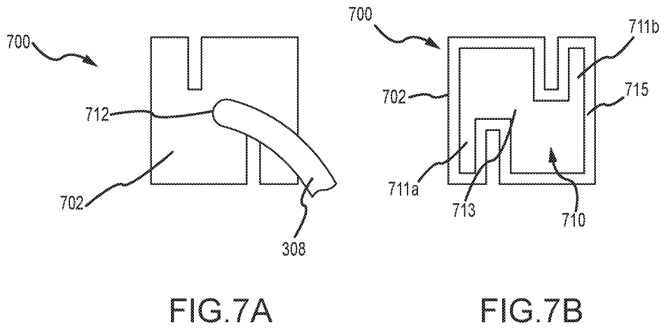

In some embodiments, the fluid collection devices described herein may be configured as a wound or other bodily fluid drainage device. FIGS. 6, 7A, and 7B show embodiments of a fluid collection devices that may be positioned adjacent to the skin on various parts of the body to remove fluid from the skin and/or wound on the user. The fluid collection devices may include fluid permeable bodies having edges and a back side covered and/or enclosed by a fluid impermeable barrier such that fluid and air may only be vacuumed or drawn into the fluid permeable barrier through a front side of the fluid permeable body. The fluid collection devices may include the tube 308 having a beveled end, thereby allowing the fluid collection devices to be used when positioned substantially horizontal on the user. In operation, when a vacuum is applied to the tube 308, the tube 308 draws fluid through the fluid permeable body into the tube 308 through the inlet. The fluid permeable body provides connections for molecular bonding of fluid in the fluid permeable body. The fluid being drawn into the tube 308 through the inlet, then, also may draw urine in the fluid permeable body 310 to the sump via the molecular bonding of the water in the urine. The configuration of the fluid permeable body and the fluid impermeable barrier may prevent a loss of vacuum on fluid being drawn from the fluid collection devices, thereby improving fluid capture from the fluid collection device.

Turning specifically to FIG. 6, which is a front view of a fluid collection device 600, according to an embodiment. The fluid collection device 600 may include a fluid impermeable barrier 602 and a fluid permeable body 610. Unless otherwise noted, the fluid impermeable barrier 602 may include any aspect of the fluid impermeable barriers 202, 302, 402, 502 and the fluid permeable body 610 may include any aspect of the fluid permeable bodies 210, 310, 410, 510.

The fluid permeable body 610 may be substantially planar or flat and include multiple side edges. For example, the fluid permeable body 610 of the fluid collection device 600 includes a generally rectangular profile and has four side edges. In FIG. 6, the side edges of the fluid permeable body 610 are covered by the side edge portions 615 of the fluid permeable body 602, and not visible. The fluid permeable body 610 may include a front side (shown in FIG. 6) and a back side (not shown). The front side of the fluid permeable body 610 is positioned to be placed at least proximate (e.g., adjacent) to the skin and/or wound of the user. In some embodiments, the front side of the fluid permeable body 610 includes a wicking material, such as gauze or other wicking fabric. The wicking material of the fluid permeable body 610 may cover spun nylon fibers material of the fluid permeable body 610 positioned between the wicking material and the back side of the fluid impermeable barrier 602.

In some embodiments, the fluid impermeable barrier 602 may include side edge portions 615 defining side slots enclosing the side edges of the fluid permeable body 610. Although the side slots defined by the side edge portions 615 and the side edges of the fluid permeable body 610 are not visible in FIG. 6, the side slots defined by the side edge portions 615 and the side edges of the fluid permeable body 610 may be similar to or the same as the slot 307 defined by the distal end region 305 of the fluid impermeable barrier 302 and the distal edge 317 of the fluid permeable body 310 shown in FIG. 3, and described above. The side edge portions 615 may enclose at least some of the front side or surface of the fluid permeable body 610 within the side slots. For example, the side edge portions 615 may cover at least some of the wicking material of the fluid permeable body. In some embodiments, the side edge portions 615 define an opening through which fluid may be drawn into the fluid collection device 600. The wicking material of the fluid permeable body 610 may extend across at least some (e.g., all) of the opening 606. The side edge portions 615 may include any of the fluid impermeable materials described above in relation to the fluid impermeable barrier 202. In some embodiments, the side edge portions 615 include a polyvinyl chloride foam. The polyvinyl chloride foam may be secured to the fluid permeable body 610 with an acrylate adhesive.

The fluid impermeable barrier 602 may cover all of the back side of the fluid permeable body 610 such that the fluid and air may be pulled into the fluid collection device 600 through the opening 606. The fluid impermeable barrier 602 also may define an aperture on the back side or an edge of the fluid collection device 600. In some embodiments, the aperture is positioned proximate to at least one side edge of the fluid collection device 600. A tube may extend through the aperture into the fluid collection device 600 and/or the aperture may otherwise provide fluid communication between the tube 308 and the fluid permeable body 610 within the fluid collection device 600. Thus, when a vacuum is drawn on the tube 308, the tube 308 draws fluid into the fluid permeable body 610 through the opening 606 and into the tube 308. The side edge portions 615 of the fluid impermeable barrier prevent air and fluid from entering the side edges of the fluid permeable body 610 to provide an improved vacuum.

FIG. 7A shows a back side view of a fluid collection device 700, and FIG. 7B shows a front side view of the fluid collection device 700, according to an embodiment. The fluid collection device 700 may include a fluid impermeable barrier 702 and a fluid permeable body 710. Unless otherwise noted, the fluid impermeable barrier 702 may include any aspect of the fluid impermeable barriers 202, 302, 402, 502, 602 and the fluid permeable body 710 may include any aspect of the fluid permeable bodies 210, 310, 410, 510, 610.

The fluid permeable body 710 may be substantially planar or flat and include multiple side edges. In some embodiments, the fluid permeable body 710 may be substantially tubular and include opposing side portions. The fluid permeable body 710 of the fluid collection device 700 may include an intermediate portion 713 and one or more arms 711a, 711b extending from the intermediate portion 713. The arms 711a, 711b may extend from opposite regions of the intermediate portion 713. In some embodiments, the fluid permeable body 710 may include a zig-zagged configuration, such as a Z-, N-, W-, M-, Y-, or X-shaped configuration with the intermediate portion 713 extending between the two arms 711a, 711b. The zig-zagged configuration increase or expand the surface area that a strip or tubular fluid permeable body 710 is able to efficiently draw fluids into the fluid permeable body 710 with a vacuum through the tube 308. Each of the intermediate portion 713 and the arms 711a, 711b may include one or more side edges or side portions. In FIG. 7B, the side edges or side portions of the intermediate portion 713 and the arms 711a, 711b are covered by the side edge portions 715 of the fluid permeable body 702, and not visible.

The fluid permeable body 710 may include a front side (shown in FIG. 7B) and a back side (covered by the fluid impermeable barrier 702 in FIG. 7A). The front side of the fluid permeable body 710 is positioned to be placed at least proximate (e.g., adjacent) to the skin and/or wound of the user. In some embodiments, the front side of the fluid permeable body 710 includes a wicking material, such as gauze or other wicking fabric. The wicking material of the fluid permeable body 710 may cover spun nylon fibers material of the fluid permeable body 710 positioned between the wicking material and the back side of the fluid impermeable barrier 702.

In some embodiments, the fluid impermeable barrier 702 may include side edge portions 715 defining side slots enclosing the side edges or side portions of the fluid permeable body 710. Although the side slots defined by the side edge portions 715 and the side edges or side portions of the fluid permeable body 710 are not visible in FIG. 7B, the side slots defined by the side edge portions 715 and the side edges or side portions of the fluid permeable body 710 may be similar to or the same as the slot 307 defined by the distal end region 305 of the fluid impermeable barrier 302 and the distal edge 317 of the fluid permeable body 310 shown in FIG. 3, and described above. The side edge portions 715 may enclose at least some of the front side or surface of the fluid permeable body 710 within the side slots. For example, the side edge portions 715 may cover at least some of the wicking material of the fluid permeable body. In some embodiments, the side edge portions 715 define an opening through which fluid may be drawn into the fluid collection device 700. The wicking material of the fluid permeable body 710 may extend across at least some (e.g., all) of the opening. The side edge portions 715 may include any of the fluid impermeable materials described above in relation to the fluid impermeable barrier 202. In some embodiments, the side edge portions 715 include a polyvinyl chloride foam. The polyvinyl chloride foam may be secured to the fluid permeable body 710 with an acrylate adhesive.

The arms 711a, 711b may be spaced from the intermediate portion 713 such that a gap is positioned between at least a portion of the side edge 715 on the arms 711a, 711b and the side edge 715 on the intermediate portion 713. In some embodiments, the side edge portions 715 of the fluid impermeable membrane 702 on the arms 711a, 711b may at least partially (e.g., entirely) interface or abut the side edge portions 715 of the intermediate portion 713. The gap or interfacing between the side edge 715 on the arms 711a, 711b and the side edge 715 on the intermediate portion 713 may extend at least partially from one terminating edge to a distal terminating edge, such as at least about one-quarter, at least about one-third, at least about one-half, at least about two-thirds, or at least about three-quarters from the one terminating edge to a distal terminating edge.

The fluid impermeable barrier 702 may cover all of the back side of the fluid permeable body 710 such that the fluid and air may be pulled into the fluid collection device 700 through the opening on the front side of the fluid collection device 700. The fluid impermeable barrier 702 also may define an aperture 712 on the back side or an edge of the fluid collection device 700. A tube may extend through the aperture 712 into the fluid collection device 700 and/or the aperture 712 may otherwise provide fluid communication between the tube 308 and the fluid permeable body 710 within the fluid collection device 700. Thus, when a vacuum is drawn on the tube 308, the tube 308 draws fluid into the fluid permeable body 710 through the opening and into the tube 308. The side edge portions 715 of the fluid impermeable barrier prevent air and fluid from entering the side edges or side portions of the fluid permeable body 710 to provide an improved vacuum.

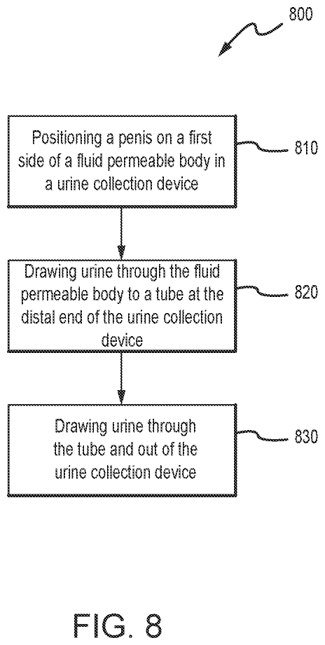

FIG. 8 is a flow diagram of a method 800 for collecting fluid from a user, according to an embodiment. The method 800 includes an act 810 of positioning a penis on a first side of a fluid permeable body in a chamber of a urine collection device. The fluid permeable body may include a second side opposite to the first side, a proximal edge, and two side edges extending from the proximal edge towards a distal end region of the urine collection device. The method also includes an act 820 of drawing urine through the fluid permeable body to a tube at the distal end region of the urine collection device. The second side, the proximal edge, and the two side edges of the fluid permeable body may be covered by a fluid impermeable barrier such that the urine is drawn through the first side of the fluid permeable body positioned against the penis. The method also includes an act 830 of drawing urine through the tube and out of the urine collection device.

In some embodiments, the method 800 also includes an act of inserting a penis through an opening in a bottom portion of the fluid impermeable barrier. A portion of the fluid impermeable barrier may cover an edge of the fluid permeable body at least partially surrounding the opening such that the penis is positioned between a top portion of the fluid impermeable barrier and the portion of the fluid impermeable barrier covering the edge of the fluid permeable body at least partially surrounding the opening.

In some embodiments, the method 800 includes an act of inserting at least a portion of the penis into a cup-shaped distal portion of the fluid permeable body at least partially defining the chamber. The fluid permeable body may include a first tab and two or more additional tabs extending from the distal portion and positioned circumferentially around the distal portion. The first tab may have the first side, the second side, the proximal edge, and the two side edges extending from the proximal edge towards the inlet. The two or more additional tabs may each have a first side, a second side covered by the fluid impermeable barrier, a proximal edge covered by the fluid impermeable barrier, and two side edges extending from the proximal edge of the additional tab towards the tube and covered by the fluid impermeable barrier.

The acts of the method 800 described above are for illustrative purposes. For example, the acts of the method 800 can be performed in different orders, split into multiple acts, modified, supplemented, or combined. In an embodiment, one or more of the act of the method 800 can be omitted from the method 800. Any of the acts of the method 800 can include using any of the fluid collection systems disclosed herein.

As used herein, the term “about” or “substantially” refers to an allowable variance of the term modified by “about” or “substantially” by +10% or +5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

1. A urine collection device, comprising:
a tube having an inlet;
a fluid permeable body having a first side, a second side, a proximal edge, and two side edges extending from the proximal edge towards the inlet;
a fluid impermeable barrier covering and interfacing substantially all of the first side, the proximal edge, and the two side edges of the fluid permeable body effective to prevent fluid communication into the fluid permeable body through the first side, the proximal edge, and the two side edges of the fluid permeable body, wherein the fluid impermeable barrier defines an aperture providing fluid communication between the fluid permeable body and at least a portion of the tube; and
a chamber sized to receive at least a portion of a penis therein to position a portion of the penis adjacent to a portion of the fluid permeable body.

2. The urine collection device of claim 1, wherein:
the fluid impermeable barrier includes a bottom portion defining an opening sized to receive at least a portion of a penis therethrough, a top portion opposite to the bottom portion, a proximal end region, a distal end region, and a proximal edge portion defining a proximal slot, the fluid impermeable barrier defining the chamber between the top portion and the bottom portion of the fluid impermeable barrier;
the fluid permeable body is positioned within the chamber and includes a distal portion positioned in or proximate to the distal end region of the fluid impermeable barrier, the proximal edge being positioned within the proximal slot such that the proximal edge interfaces the proximal edge portion of the fluid impermeable barrier; and
the tube extends into the chamber and the inlet is positioned within the chamber proximate to the distal end region of the fluid impermeable barrier.

3. The urine collection device of claim 2, wherein:
the fluid impermeable barrier includes an additional edge portion defining an additional slot surrounding the opening such that the fluid impermeable barrier extends continuously from the bottom portion of the fluid impermeable barrier, through the opening, and at least partially into the chamber; and
the fluid permeable body includes an additional edge positioned within the additional slot such that the additional edge interfaces the additional edge portion of the fluid impermeable barrier and surrounds at least a portion of the opening.

4. The urine collection device of claim 2, wherein:
the fluid impermeable barrier includes a first side edge portion defining a first side slot and a second side edge portion defining a second slot, each of the first side slot and the second side slot extending at least partially between the proximal edge portion and the distal end region of the chamber; and
the two side edges of the fluid permeable body include a first side edge positioned within the first side slot such that the first side edge interfaces the first side edge portion and a second side edge positioned within the second side slot such that the second side edge interfaces the second side edge portion.

5. The urine collection device of claim 2, wherein the distal portion of fluid permeable body covers the inlet.

6. The urine collection device of claim 1, wherein:
the fluid permeable body includes:
a cup-shaped distal portion having a first side and a second side, the second side at least partially defining the chamber sized to receive at least the portion of the penis therein to position the portion of the penis adjacent to the distal portion of the fluid permeable body;
a first tab extending from the distal portion and having the first side, the second side, the proximal edge, and the two side edges extending from the proximal edge towards the inlet; and
two or more additional tabs each extending from the distal portion and having a first side, a second side, a proximal edge, and two side edges extending from the proximal edge of the additional tab towards the inlet, the first tab and the two or more additional tabs positioned circumferentially around the distal portion;
the fluid impermeable barrier covers:
the first side, the proximal edge, and the two side edges of each of the first tab and the two or more additional tabs of the fluid permeable body effective to prevent fluid communication through the first side, the proximal edge, and the two side edges of each of the first tab and the two or more additional tabs; and
the first side of the distal portion of the fluid permeable body.

7. The urine collection device of claim 6, wherein the fluid impermeable barrier covers at least a portion of the second side of each of the first tab and the two or more additional tabs proximate to the proximal edge and the two side edges of each of the first tab and the two or more additional tabs.

8. The urine collection device of claim 1, wherein the fluid permeable body includes a fluid permeable porous fibers structure and an outer fluid permeable wicking material.

9. A fluid collection device, comprising:
a substantially planar fluid permeable body having a first side, a second side, and one or more side edges;
a fluid impermeable barrier defining an aperture and covering and interfacing substantially all of the first side and the one or more side edges of the fluid permeable body; and
a tube extending through the aperture and having an inlet at least partially covered by the fluid permeable barrier,
wherein the fluid permeable body includes an intermediate portion having one or more side edges and multiple arms extending from the intermediate portion each having one or more side edges, the fluid impermeable barrier covering the one or more side edges of the intermediate portion and the multiple arms of the fluid permeable body.

10. (canceled)

11. (canceled)

12. The fluid collection device of claim 9, wherein the multiple arms included elongated arms angled relative to one another.

13. The fluid collection device of claim 9, wherein the multiple arms include two arms substantially parallel to one another with the intermediate portion positioned between the two arms.

14. A method of collecting urine, the method comprising:
positioning a penis on a first side of a fluid permeable body in a chamber of a urine collection device, the fluid permeable body having a second side opposite to the first side, a proximal edge, and two side edges extending from the proximal edge towards a distal end region of the urine collection device;
drawing urine through the fluid permeable body to an inlet of a tube at the distal end region of the urine collection device, wherein substantially all of the second side, the proximal edge, and the two side edges of the fluid permeable body are covered and interfaced by a fluid impermeable barrier such that the urine is drawn only through the first side of the fluid permeable body positioned against the penis; and
drawing at least some of the urine through the inlet of the tube and out of the urine collection device.

15. The method of claim 14, further comprising inserting a penis through an opening in a bottom portion of the fluid impermeable barrier.

16. The method of claim 15, wherein inserting a penis through an opening in a bottom portion of the fluid impermeable barrier includes inserting the penis through the opening in the bottom portion of the fluid impermeable barrier, a portion of the fluid impermeable barrier covering an edge of the fluid permeable body at least partially surrounding the opening such that the penis is positioned between a top portion of the fluid impermeable barrier and the portion of the fluid impermeable barrier covering the edge of the fluid permeable body at least partially surrounding the opening.

17. The method of claim 14, further comprising inserting at least a portion of the penis into a cup-shaped distal portion of the fluid permeable body at least partially defining the chamber, the fluid permeable body including a first tab and two or more additional tabs extending from the distal portion and positioned circumferentially around the distal portion, the first tab having the first side, the second side, the proximal edge, and the two side edges extending from the proximal edge towards the inlet, the two or more additional tabs each having a first side, a second side covered by the fluid impermeable barrier, a proximal edge covered by the fluid impermeable barrier, and two side edges extending from the proximal edge of the additional tab towards the tube and covered by the fluid impermeable barrier.

18. The method of claim 14, wherein the fluid permeable body includes a fluid permeable porous fiber structure and an outer fluid permeable wicking material.
